(19) United States
(12) Patent Application Publication
(10) Pub. No.: US 2006/0172297 A1
(43) Pub. Date: Aug. 3, 2006

(54) Enhancers specific to motor nerve cells/sensory nerve cells

(75) Inventors: Hitoshi Okamoto, Saitama (JP); Osamu Uemura, Saitama (JP); Shin-Ichi Higashijima, New York, NY (US)

Correspondence Address: GREENBLUM & BERNSTEIN, P.L.C., 1950 ROLAND CLARKE PLACE, RESTON, VA, 20191, US

(73) Assignee: RIKEN, Salitama 351-0198 (JP)

(21) Appl. No.: 10/525,725

(22) PCT Filed: Aug. 29, 2003
(86) PCT No.: PCT/JP03/11076

§ 371 Date: Feb. 28, 2005

(30) Foreign Application Priority Data
Aug. 30, 2002 (JP) 2002-254829

(51) Int. Cl. C12Q 1/68; C07H 21/04; C12N 5/08; C12N 15/87

(52) U.S. Cl. 435/6; 435/455; 435/368; 536/23.2

(57) ABSTRACT

This invention provides a method for improving gene expression efficiency specifically in motor neurons and/or in sensory neurons and an enhancer consisting of the following DNA (a), (b), or (c): (a) DNA consisting of the nucleotide sequence as shown in any one of SEQ ID NOs: 1 to 4; (b) DNA consisting of a nucleotide sequence derived from the nucleotide sequence as shown in any one of SEQ ID NOs: 1 to 4 by deletion, substitution, or addition of one or more nucleotides and capable of improving gene expression efficiency in motor neurons; or (c) DNA consisting of a nucleotide sequence capable of hybridizing under stringent conditions to a nucleotide sequence complementary to the nucleotide sequence as shown in any one of SEQ ID NOs: 1 to 4 and capable of improving gene expression efficiency in motor neurons.

### TECHNICAL FIELD

The present invention relates to an enhancer that can enhance expression of a gene of interest specifically in motor neurons and/or in sensory neurons.

### BACKGROUND ART

Arber et al. (Neuron, 1999, August 23: 659-674) isolated the mouse Hb9 gene promoter that would be expressed specifically in motor neurons. This promoter is known to induce reporter gene expression specifically in somatic motor neurons of a transgenic mouse. However, the Hb9 gene expression in the motor neurons is transient, and it is considered difficult to drive reporter gene expression in mature cells. Further, this system does not drive reporter gene expression in visceral motor neurons. This promoter contains a region approximately 9 kbp upstream from the transcription initiation site of the Hb9 gene, and this promoter is deduced to involve complication in handling at the time of operations such as preparation of a recombinant. Since this promoter is derived from a mouse, some ethical concerns may arise if it is used for treatment.

An enhancer is a specific nucleotide sequence that improves the efficiency of transcription by RNA polymerase. An enhancer is located upstream or downstream of a specific gene and capable of accelerating transcription of a specific gene when a regulatory protein or the like capable of regulating gene expression is bound thereto.

Given the circumstances, the present invention is directed to providing an enhancer that can improve gene expression efficiency specifically in motor neurons and/or in sensory neurons. Also, the present invention is directed to providing a vector comprising such enhancer, a transgenic animal, and a method for regulating gene expression. Further, the present invention is directed to providing a method for evaluating differentiation of pluripotent stem cells using such enhancer and a method for regenerating motor neurons and/or sensory neurons.

### DISCLOSURE OF THE INVENTION

The present invention, whereby the above objects have been accomplished, includes the following features.

(1) An enhancer consisting of the following DNA (a), (b), or (c):
(a) DNA consisting of the nucleotide sequence as shown in any one of SEQ ID NOs: 1 to 4;
(b) DNA consisting of a nucleotide sequence derived from the nucleotide sequence as shown in any one of SEQ ID NOs: 1 to 4 by deletion, substitution, or addition of one or more nucleotides and capable of enhancing gene expression efficiency in motor neurons; or
(c) DNA consisting of a nucleotide sequence capable of hybridizing under stringent conditions to a nucleotide sequence complementary to the nucleotide sequence as shown in any one of SEQ ID NOs: 1 to 4 and capable of enhancing gene expression efficiency in motor neurons.

(2) An enhancer consisting of the following DNA (a), (b), or (c):
(a) DNA consisting of any one of a nucleotide sequence consisting of nucleotides 235 to 560 of SEQ ID NO: 1, a nucleotide sequence consisting of nucleotides 204 to 528 of SEQ ID NO: 2, a nucleotide sequence consisting of nucleotides 206 to 530 of SEQ ID NO: 3, or a nucleotide sequence consisting of nucleotides 211 to 555 of SEQ ID NO: 4;
(b) DNA consisting of a nucleotide sequence derived from any one of a nucleotide sequence consisting of nucleotides 235 to 560 of SEQ ID NO: 1, a nucleotide sequence consisting of nucleotides 204 to 528 of SEQ ID NO: 2, a nucleotide sequence consisting of nucleotides 206 to 530 of SEQ ID NO: 3, or a nucleotide sequence consisting of nucleotides 211 to 555 of SEQ ID NO: 4 by deletion, substitution, or addition of one or more nucleotides and capable of enhancing gene expression efficiency in motor neurons; or
(c) DNA consisting of a nucleotide sequence capable of hybridizing under stringent conditions to a nucleotide sequence complementary to any one of a nucleotide sequence consisting of nucleotides 235 to 560 of SEQ ID NO: 1, a nucleotide sequence consisting of nucleotides 204 to 528 of SEQ ID NO: 2, a nucleotide sequence consisting of nucleotides 206 to 530 of SEQ ID NO: 3, or a nucleotide sequence consisting of nucleotides 211 to 555 of SEQ ID NO: 4 and capable of enhancing gene expression efficiency in motor neurons.

(3) The enhancer according to (1) or (2), wherein the motor neurons dorsally extend axons.

(4) An enhancer consisting of the following DNA (a), (b), or (c):
(a) DNA consisting of the nucleotide sequence as shown in SEQ ID NO: 5;
(b) DNA consisting of a nucleotide sequence derived from the nucleotide sequence as shown in SEQ ID NO: 5 by deletion, substitution, or addition of one or more nucleotides and capable of enhancing gene expression efficiency in sensory neurons; or
(c) DNA consisting of a nucleotide sequence capable of hybridizing under stringent conditions to a nucleotide sequence complementary to the nucleotide sequence as shown in SEQ ID NO: 5 and capable of enhancing gene expression efficiency in sensory neurons.

(5) An enhancer consisting of the following DNA (a), (b), or (c):
(a) DNA consisting of the nucleotide sequence as shown in SEQ ID NO: 5 or 6;
(b) DNA consisting of a nucleotide sequence derived from the nucleotide sequence as shown in SEQ ID NO: 5 or 6 by deletion, substitution, or addition of one or more nucleotides and capable of enhancing gene expression efficiency in motor neurons that ventrally extend axons; or
(c) DNA consisting of a nucleotide sequence capable of hybridizing under stringent conditions to a nucleotide sequence complementary to the nucleotide sequence as shown in SEQ ID NO: 5 and capable of enhancing gene expression efficiency in motor neurons that ventrally extend axons.

(6) A vector comprising the enhancer according to any one of (1) to (5).

(7) The vector according to (6) further comprising a promoter and a gene comprising a coding region.

(8) A transgenic cell line comprising the vector according to (6) or (7).

(9) A transgenic animal comprising the vector according to (6) or (7).

(10) A method for regulating gene expression, wherein expression efficiency of a given gene is improved under the control of the enhancer according to any one of (1) to (5).

(11) A method for regulating gene expression comprising a step of introducing a nucleic acid construct comprising the enhancer according to any one of (1) to (5) and the given gene into cells, wherein expression efficiency of the given gene is improved in motor neurons and/or in sensory neurons.

(12) A method for evaluating differentiation of pluripotent stem cells comprising a step of introducing a nucleic acid construct comprising the enhancer according to any one of (1) to (5), a promoter, and a reporter gene into pluripotent stem cells and a step of inducing the pluripotent stem cells to differentiate, wherein reporter gene expression is assayed to evaluate whether or not the pluripotent stem cells are differentiated into motor neurons or sensory neurons.

(13) A method for regenerating motor neurons/sensory neurons comprising steps of:
introducing a nucleic acid construct comprising the enhancer according to any one of (1) to (5), a promoter, and a reporter gene into pluripotent stem cells;
inducing the pluripotent stem cells to differentiate;
assaying reporter gene expression to evaluate whether or not the pluripotent stem cells are differentiated into motor neurons or sensory neurons to thereby select motor neurons or sensory neurons; and
transplanting the selected motor neurons or sensory neurons.

This description includes part or all of the contents as disclosed in the description and/or drawings of Japanese Patent Application No. 2002-254829, which is a priority document of the present application.

### BRIEF DESCRIPTION OF THE DRAWINGS

FIG. 1 shows an EcoRI restriction map of the vicinity of the Islet-1 gene of the zebrafish genome.

FIG. 2 shows a schematic illustration of an ICP-GFP plasmid.

FIG. 3 shows the design of a plasmid comprising a partial fragment of the CM region.

FIG. 4 shows a schematic illustration of an hsp-GFP plasmid.

FIG. 5 is a photograph showing a dorsal view of the head of a zebrafish to which CMB3-hsp-GFP has been injected.

FIG. 6 is a photograph showing a dorsal view of the head of a zebrafish to which CM-ICP-GFP has been injected.

FIG. 7 shows the design of a plasmid comprising a partial fragment of the SS region.

FIG. 8 is a photograph showing a lateral view of the head of a zebrafish to which SSd25/30-hsp-GFP has been injected.

FIG. 9 is a photograph showing a lateral view of the spinal cord of a zebrafish to which SSd25/30-hsp-GFP has been injected.

FIG. 10 is a photograph showing a lateral view of the head of a zebrafish to which SS-hsp-GFP has been injected.

FIG. 11 is a photograph showing a lateral view of the spinal cord of a zebrafish to which SS-hsp-GFP has been injected.

FIG. 12 is a photograph showing a lateral view of the head of a zebrafish to which SS-ICP-GFP has been injected.

FIG. 13 is a photograph showing a lateral view of the spinal cord of a zebrafish to which SS-ICP-GFP has been injected.

FIG. 14 shows the alignment results of a homology search among zCM, huCM, mCM, and fuguCM.

FIG. 15 is a photograph showing a dorsal view of the head of a zebrafish to which zCM-ICP-GFP, huCM-ICP-GFP, or mCM-ICP-GFP has been injected.

FIG. 16 is a photograph showing a lateral view of the spinal cord of a zebrafish to which huCM-ICP-GFP or mCM-ICP-GFP has been injected.

FIG. 17 shows the alignment results of a homology search between zSS and huSS.

FIG. 18 is a photograph showing a lateral view of the head and that of the spinal cord of a transgenic zebrafish generated with the use of zSS-ICP-GFP.

FIG. 19 is a photograph showing a lateral view of the brain of a transgenic mouse generated with the use of zCM-ICP-PLAP.

FIG. 20 is a photograph showing the anterior view of the brain of a transgenic mouse generated with the use of zCM-ICP-PLAP.

FIG. 21 is a photograph showing a lateral view of the ribs of a transgenic mouse generated with the use of zCM-ICP-PLAP.

FIG. 22 is a photograph showing the inside view of the ribs of a transgenic mouse generated with the use of zCM-ICP-PLAP.

FIG. 23 is a photograph showing a lateral view of the head of a transgenic mouse generated with the use of zSS-ICP-PLAP.

FIG. 24 is a photograph showing a transgenic zebrafish generated with the use of SS-hsp-GFP and a transgenic mouse generated with the use of zSS-ICP-PLAP and huSS-ICP-PLAP.

FIG. 25 is a photograph showing a transgenic zebrafish generated with the use of CM-ICP-GFP and a transgenic mouse prepared with the use of huCM-ICP-PLAP.

### BEST MODES FOR CARRYING OUT THE INVENTION

Hereafter, the present invention is described in detail.

The enhancer according to the present invention consists of: (a) the nucleotide sequence as shown in SEQ ID NO: 1; (b) a nucleotide sequence derived from the nucleotide sequence as shown in SEQ ID NO: 1 by deletion, substitution, or addition of one or more nucleotides and capable of enhancing gene expression efficiency in motor neurons; or (c) a nucleotide sequence capable of hybridizing under stringent conditions to a nucleotide sequence complementary to the nucleotide sequence as shown in SEQ ID NO: 1 and capable of enhancing gene expression efficiency in motor neurons.

The nucleotide sequence as shown in SEQ ID NO: 1 is contained in a region (hereafter, referred to as the “CM region”) located approximately 10 kbp downstream of the transcription initiation site of the Islet-1 gene of the zebrafish genome. A DNA fragment consisting of the nucleotide sequence as shown in SEQ ID NO: 1 is an enhancer that is capable of enhancing gene expression efficiency specifically in the motor neuron. The Islet-1 gene is an early marker for differentiated motor neurons, and it is expressed in motor neurons and in sensory neurons in the case of a 24-hour post fertilization (hpf) zebrafish embryo.

The enhancer according to the present invention consists of: (a) the nucleotide sequence as shown in SEQ ID NO: 2, 3, or 4; (b) a nucleotide sequence derived from the nucleotide sequence as shown in SEQ ID NO: 2, 3, or 4 by deletion, substitution, or addition of one or more nucleotides and capable of enhancing gene expression efficiency in motor neurons; or (c) a nucleotide sequence capable of hybridizing under stringent conditions to a nucleotide sequence complementary to the nucleotide sequence as shown in SEQ ID NO: 2, 3, or 4 and capable of enhancing gene expression efficiency in motor neurons.

The nucleotide sequence as shown in SEQ ID NO: 2 can be identified by conducting a homology search of the human genome database that is disclosed by the National Center for Biotechnology Information (NCBI), based on the nucleotide sequence as shown in SEQ ID NO: 1. Also, the nucleotide sequence as shown in SEQ ID NO: 3 can be identified by conducting a homology search of the mouse genome database that is disclosed by NCBI, based on the nucleotide sequence as shown in SEQ ID NO: 1. Further, the nucleotide sequence as shown in SEQ ID NO: 4 can be identified by conducting a homology search of the fugu genome database that is disclosed by NCBI, based on the nucleotide sequence as shown in SEQ ID NO: 1.

Specifically, the nucleotide sequence as shown in SEQ ID NO: 1 is a zebrafish genome-derived enhancer, the nucleotide sequence as shown in SEQ ID NO: 2 is a human genome-derived enhancer, the nucleotide sequence as shown in SEQ ID NO: 3 is a mouse genome-derived enhancer, and the nucleotide sequence as shown in SEQ ID NO: 4 is a fugu genome-derived enhancer.

In the following description, (a) the enhancer consisting of DNA consisting of the nucleotide sequence as shown in any one of SEQ ID NOs: 1 to 4; (b) the enhancer consisting of DNA consisting of a nucleotide sequence derived from the nucleotide sequence as shown in any one of SEQ ID NOs: 1 to 4 by deletion, substitution, or addition of one or more nucleotides and capable of enhancing gene expression efficiency in motor neurons; and (c) the enhancer consisting of DNA consisting of a nucleotide sequence capable of hybridizing under stringent conditions to a nucleotide sequence complementary to the nucleotide sequence as shown in any one of SEQ ID NOs: 1 to 4 and capable of enhancing gene expression efficiency in motor neurons are collectively referred to as “CM enhancers.”

Meantime, the enhancer according to the present invention consists of: (a) the nucleotide sequence as shown in SEQ ID NO: 5; (b) a nucleotide sequence derived from the nucleotide sequence as shown in SEQ ID NO: 5 by deletion, substitution, or addition of one or more nucleotides and capable of enhancing gene expression efficiency in sensory neurons and in motor neurons that ventrally extend axons; or (c) a nucleotide sequence capable of hybridizing under stringent conditions to a nucleotide sequence complementary to the nucleotide sequence as shown in SEQ ID NO: 5 and capable of enhancing gene expression efficiency in sensory neurons and in motor neurons that ventrally extend axons.

The nucleotide sequence as shown in SEQ ID NO: 5 is contained in a region (hereafter, referred to as the “SS region”) located approximately 55 kbp downstream of the transcription initiation site of the Islet-1 gene of the zebrafish genome. A DNA fragment consisting of the nucleotide sequence as shown in SEQ ID NO: 5 is an enhancer that is capable of enhancing gene expression efficiency specifically in the sensory neuron nuclei and in motor neurons that extend axons ventrally.

The nucleotide sequence as shown in SEQ ID NO: 6 can be identified by conducting a homology search of the human genome database that is disclosed by the National Center for Biotechnology Information (NCBI), based on the nucleotide sequence as shown in SEQ ID NO: 5. Accordingly, the enhancer according to the present invention consists of: (a) the nucleotide sequence as shown in SEQ ID NO: 6; (b) a nucleotide sequence derived from the nucleotide sequence as shown in SEQ ID NO: 6 by deletion, substitution, or addition of one or more nucleotides and capable of enhancing gene expression efficiency in motor neurons that extend axons ventrally; or (c) a nucleotide sequence capable of hybridizing under stringent conditions to a nucleotide sequence complementary to the nucleotide sequence as shown in SEQ ID NO: 6 and capable of enhancing gene expression efficiency in motor neurons that extend axons ventrally. The enhancer consisting of the nucleotide sequence as shown in SEQ ID NO: 6 does not exhibit activity in sensory neurons.

In the following description, (a) the enhancer consisting of DNA consisting of the nucleotide sequence as shown in SEQ ID NO: 5 or 6; (b) the enhancer consisting of DNA consisting of a nucleotide sequence derived from the nucleotide sequence as shown in SEQ ID NO: 5 or 6 by deletion, substitution, or addition of one or more nucleotides and capable of enhancing gene expression efficiency in sensory neurons and/or in motor neurons that extend axons ventrally; and (c) the enhancer consisting of DNA consisting of a nucleotide sequence capable of hybridizing under stringent conditions to a nucleotide sequence complementary to the nucleotide sequence as shown in SEQ ID NO: 5 or 6 and capable of enhancing gene expression efficiency in sensory neurons and/or in motor neurons that extend axons ventrally are collectively referred to as “SS enhancers.”

A nucleotide sequence derived from the nucleotide sequences as shown in any of SEQ ID NOs: 1 to 6 by deletion, substitution, or addition of one or more nucleotides refers to a nucleotide sequence with deletion, substitution, or addition of, for example, 1 to 30 nucleotides, preferably 1 to 20 nucleotides, and more preferably 1 to 10 nucleotides. In particular, a region to be deleted, substituted, or added is preferably a region excluding the region between nucleotides 235 and 560 of SEQ ID NO: 1, a region excluding the region between nucleotides 204 and 528 of SEQ ID NO: 2, a region excluding the region between nucleotides 206 and 530 of SEQ ID NO: 3, a region excluding the region between nucleotides 211 and 555 of SEQ ID NO: 4, a region excluding the region between nucleotides 378 and 553 of SEQ ID NO: 5, or a region excluding the region between nucleotides 178 and 353 of SEQ ID NO: 6.

The region between nucleotides 235 and 560 of SEQ ID NO: 1, the region between nucleotides 204 and 528 of SEQ ID NO: 2, the region between nucleotides 206 and 530 of SEQ ID NO: 3, and the region between nucleotides 211 and 555 of SEQ ID NO: 4 are highly homologous to one another and are highly conserved among species. Accordingly, it is suggested that these regions of SEQ ID NOs: 1 to 4 contribute to functions of enhancers for improving gene expression efficiency in motor neurons. The region between nucleotides 378 and 553 of SEQ ID NO: 5 and the region between nucleotides 178 and 353 of SEQ ID NO: 6 are highly homologous to each other and are highly conserved among species. Accordingly, it is suggested that these regions of SEQ ID NOs: 5 and 6 contribute to functions of enhancers for improving gene expression efficiency in sensory neurons and/or in motor neurons that extend axons ventrally.

When one or more nucleotides are deleted from, substituted, or added to the nucleotide sequence as shown in any of SEQ ID NOs: 1 to 6, any conventional techniques can be adequately employed without particular limitation. For example, a given nucleotide can be substituted via site-directed mutagenesis. Examples of site-directed mutagenesis include site-directed mutagenesis of T. Kunkel (Kunkel, T. A., Proc. Nati, Acad. Sci., U.S.A., 82, pp 488-492, 1985) and the gapped duplex method. Also, a modification method for efficiently conducting substitution at multiple sites can be employed. In such a technique, a maximal of 16 oligonucleotides may be simultaneously used whereas 1 or 2 modification oligonucleotides are used in the common Kunkel method. In the present invention, mutation can be introduced using a mutagenesis kit via site-directed mutagenesis (e.g., Mutan-K or Mutan-G, Takara Shuzo Co., Ltd.) or the LA PCR in vitro Mutagenesis series kit (Takara Shuzo Co., Ltd.).

The phrase “hybridizing under stringent conditions to a nucleotide sequence complementary to the nucleotide sequence as shown in any one of SEQ ID NOs: 1 to 6” refers to the fact that a what is called specific hybrid is formed but a non-specific hybrid is not formed under stringent conditions. For example, highly homologous nucleic acids, i.e., DNAs that exhibit 60% or higher, and preferably 80% or higher homology are hybridized to each other, but DNAs that exhibit homology lower than the aforementioned levels are not hybridized to each other. Under stringent conditions, for example, hybridization takes place at a sodium concentration of 15 mM to 900 mM, and at a temperature of 37° C. to 70° C. (preferably 68° C.).

The term “functions for improving gene expression efficiency in motor neurons and/or in sensory neurons” refers to the fact that functions for improving transcription efficiency of a gene of interest are developed particularly specifically in motor neurons and/or in sensory neurons. Whether or not a DNA fragment comprising an arbitrary nucleotide sequence has such functions can be determined in the following manner. The DNA fragment is incorporated into an expression vector comprising an arbitrary promoter and a reporter gene to prepare a recombinant vector, and a transgenic animal is prepared using the recombinant vector. Thereafter, whether or not the expression level of a reporter gene in motor neurons and/or in sensory neurons of the transgenic animal is elevated is inspected. When the reporter gene expression in motor neurons and/or in sensory neurons of the transgenic animal is elevated, the DNA fragment of interest can be determined to have the aforementioned functions.

The CM enhancers exhibit enhancer activities particularly in motor neurons that extend axons dorsally among various types of motor neurons, and they also exhibit transient enhancer activities in all motor neurons. The SS enhancers exhibit enhancer activities specifically in motor neurons that extend axons ventrally among various types of motor neurons. Although the zebrafish-derived SS enhancers exhibit enhancer activities in sensory neurons, the human-derived SS enhancers do not exhibit enhancer activities in sensory neurons.

The species of animal for generating transgenic animals is not particularly limited. However, a transgenic animal in which reporter gene expression in motor neurons and/or in sensory neurons can be easily determined is preferable. Examples of transgenic animals that can be employed include a transgenic zebrafish and a transgenic mouse.

Alternatively, the aforementioned recombinant vector is injected into a one-cell stage zebrafish embryo via microinjection or other means, and reporter gene expression in the embryo is confirmed 24 hours thereafter. Thus, whether or not a DNA fragment of interest has functions for improving gene expression efficiency in motor neurons and/or in sensory neurons can be confirmed.

Examples of reporter genes that can be employed in the present invention include a green fluorescent protein (GFP) gene encoding a green fluorescent protein, an alkaline phosphatase gene, and a β-galactosidase (lacZ) gene.

Examples of promoters that can be employed include a zebrafish-derived Islet-1 promoter located approximately 4 kbp upstream of the Islet-1 gene (the Islet-1 core promoter, hereafter referred to as “ICP”), a zebrafish-derived heat shock 70 promoter, and a human-derived promoter. A specific example thereof is a β-globin promoter.

An expression vector that can increase the expression level of a gene of interest specifically in motor neurons or in sensory neurons can be constructed using the enhancer according to the present invention. A variety of promoters mentioned above can be employed. When preparation of an expression vector for human gene therapy is intended, use of a human-derived promoter, i.e., a β-globin promoter, is particularly preferable.

Genes, the expression level of which is intended to increase specifically in motor neurons or in sensory neurons, are not particularly limited. An example thereof is the Bc1-2 proto-oncogene (Science, 1997, July 25, 277, 559-562) that is known to prolong the survival of a patient group having familial amyotrophic lateral sclerosis (ALS) who has mutation in the SOD1 gene via overexpression thereof. When gene therapy is intended with the use of an expression vector having the enhancer according to the present invention, a promoter, and the Bc1-2 proto-oncogene, Bc1-2 can be expressed specifically in motor neurons. Thus, an effect of improving the vital prognosis of a patient with familial ALS can be expected.

An example of a gene the expression level of which is intended to increase specifically in sensory neurons is the preproenkephalin gene. Preproenkephalin is a precursor of an endogenous narcotic-like substance, i.e., enkephalin. Induction of overexpression thereof in sensory neurons may lead to alleviation of chronic pain from, for example, chronic rheumatism (Braz J. et al., J. Neurosci 2001, October 15, 21 (20), 7881-8).

The aforementioned enhancer according to the present invention can be utilized in a method of evaluating differentiation of pluripotent stem cells. In this case, a nucleic acid construct comprising the enhancer, a promoter, and a reporter gene is first prepared. The “nucleic acid construct” may be the aforementioned expression vector comprising the enhancer, the promoter, and the reporter gene. The term “pluripotent stem cells” refers to stem cells that are capable of differentiating into all the tissue cells constituting an individual organism and are present at the early stage of development, i.e., stem cells having pluripotency. An example of a pluripotent stem cell is an embryonic stem cell (ES cell) that has been established to select a stem cell having pluripotency, which are present at the early stage of development, and to be then cultured in vitro.

A method of introducing a nucleic acid construct into a pluripotent stem cell is not particularly limited. Examples thereof include injection utilizing a microscope and electroporation.

Subsequently, the pluripotent stem cell to which a nucleic acid construct has been introduced is induced to differentiate into motor neurons or sensory neurons. An example of a method for inducing differentiation is culture using a culture solution containing retinoic acid.

Thereafter, reporter gene expression in the differentiated pluripotent stem cell is assayed. Specifically, cells in which the level of reporter gene expression is significantly increased compared with that in the control can be determined to be cells that have been differentiated into motor neurons motor neurons that have been differentiated from a pluripotent stem cell can be easily and accurately selected by selecting cells in which the level of reporter gene expression has been significantly increased.

Further, the selected motor neurons can be employed for what is called “regenerative medicine.” More specifically, the selected motor neurons are further cultured and then transplanted to a control animal such as a human. Thus, an effective method of treatment can be provided for patients who have lost motor nerves.

In contrast, the enhancer according to the present invention can be employed for what is called “gene therapy.” More specifically, a nucleic acid construct that comprises a gene to be introduced so as to be regulated by the aforementioned enhancer is prepared. A nucleic acid construct is not particularly limited as long as it can be employed for gene therapy. Examples thereof include an adenovirus vector, a herpes virus vector, and a retrovirus vector comprising the aforementioned enhancer, a promoter, and a gene to be introduced.

A gene to be introduced is not particularly limited. A gene that is known to exhibit therapeutic and ameliorating effects on a given disease by being expressed specifically in motor neurons and/or in sensory neurons can be employed. Examples thereof include the proto-oncogene Bc1-2 (Science, 1997, July 25, 277, 559-562) and the preproenkephalin gene as mentioned above.

Subsequently, gene therapy is provided for the target patient using a nucleic acid construct in accordance with a conventional technique.

Thus, the gene to be introduced can be expressed specifically in motor neurons or in sensory neurons of the patient who had received gene therapy as mentioned above. With the gene therapy utilizing the enhancer according to the present invention, the gene to be introduced can be expressed specifically in motor neurons or in sensory neurons. Accordingly, more effective therapeutic and ameliorating effects can be expected.

Hereafter, the present invention is described in greater detail with reference to the following examples, although the technical scope of the present invention is not limited thereto.

### EXAMPLE 1

### Identification of Enhancer Region

At the outset, a region exhibiting enhancer functions contained in the CM region located approximately 10 kbp downstream of the transcription initiation site of the Islet-1 gene and that in the SS region located approximately 55 kbp downstream thereof were identified in the following manner.

i) Identification of Islet-1 Gene Promoter

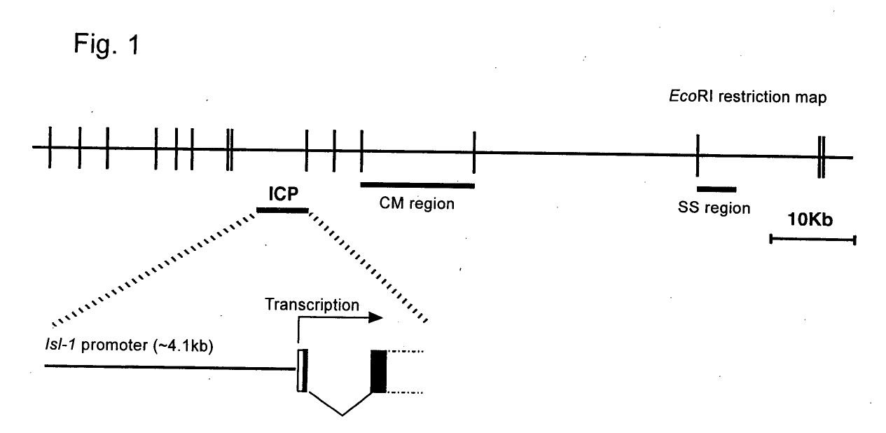

The zebrafish genome library was screened via Southern hybridization utilizing Islet-1 cDNA as a probe, and a full-length Islet-1 gene and a positive clone containing the genome sequence (approximately 4.1 kbp) located upstream thereof were isolated. This positive clone was used as a template to isolate ICP using a standard PCR technique (see FIG. 1).

Specifically, the zebrafish genome library was prepared using λ DASH II as a phage vector in accordance with a conventional technique. A sequence (ICP 5 prime) derived from a phage vector and a sequence (ICP 3 prime) containing the 5′-non-translated region of the Islet-1 gene to which the restriction enzyme KpnI recognition sequence had been added at its 3′ end were used as primers in the aforementioned PCR.

The primer sequences are as shown below.

| | |
|---|---|
| ICP 5 prime; | |
| GAATTCGGATCCAAGGTCTTCAGTCT | (SEQ ID NO: 7) |
| ICP 3 prime; | |
| GGTACCTGTATTGATGGGCCAC | (SEQ ID NO: 8) |

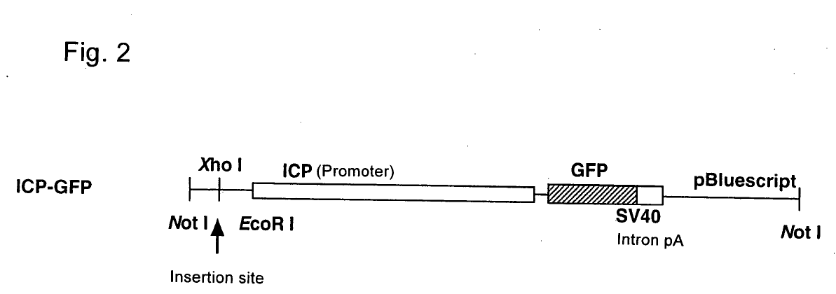

Whether the region amplified using the aforementioned PCR was capable of functioning as a promoter or not was confirmed in the following manner. Specifically, pBluescript II SK (Stratagene) was first constructed. In this construct, the green fluorescent protein (GFP) to which the intron/poly A additional signal derived from the SV 40 virus had been ligated was ligated as a reporter gene to a position immediately downstream of the DNA fragment amplified using PCR (see FIG. 2). The constructed plasmid was designated as ICP-GFP.

ICP-GFP was microinjected into one-cell stage zebrafish embryos. Specifically, a holder to which a plastic syringe has been connected and a glass micropipette were used for microinjection. A DNA solution adjusted to approximately 25 ng/microlitter was placed in the glass micropipette, and the solution was injected in a manner such that the solution was spread over approximately one third of the diameter of the cytoplasm under a stereoscopic microscope (Zeiss). After the microinjection, the zebrafish was raised under normal conditions, and the GFP expression in the 24 hpf embryos was assayed under a fluorescent stereoscopic microscope (Leica).

As a result, the Islet-1 gene promoter was identified and isolated.

ii) Identification of CM Region and SS Region

A clone (approximately 100 kbp) containing the Islet-1 gene was isolated from the zebrafish BAC library using Islet-1 cDNA as a template.

The isolated clone of approximately 100 kbp containing the Islet-1 gene was completely digested with the restriction enzyme EcoRI, and the resulting fragments were inserted into upstream of the ICP of ICP-GFP. The ICP-GFP containing the EcoRI-digested BAC clone fragment was microinjected into one-cell stage zebrafish embryos, and GFP-induced fluorescence expression was examined by the transient expression assay.

As a result, a genome fragment (approximately 15 kbp) that drove GFP expression specifically in the cranial motor neurons was identified. This genome fragment was located approximately 10 kbp downstream of the transcription initiation site of the Islet-1 gene. This genome fragment was designated as the CM region (FIG. 1).

Also, a genome fragment (approximately 4.5 kbp) that induced GFP expression specifically in sensory neurons was identified. This genome fragment was located approximately 55 kbp downstream of the transcription initiation site of the Islet-1 gene. This genome fragment was designated as the SS region (FIG. 1).

iii) Analysis of Enhancer Contained in CM Region and SS Region

The minimal regions that exhibited enhancer activities of the CM region and the SS region identified in ii) above were analyzed.

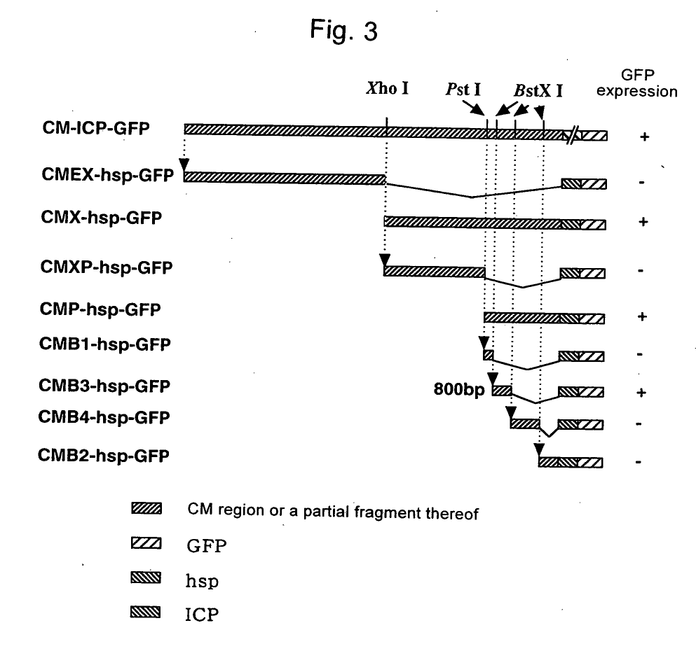

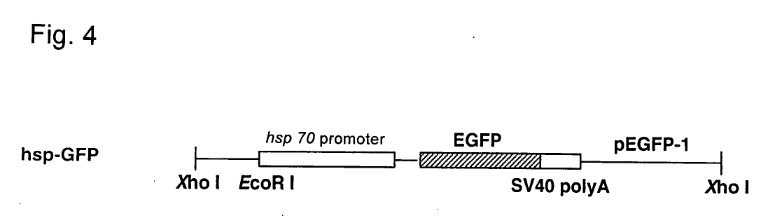

As shown in FIG. 3, partial fragments of the CM region were designed using the XhoI, PstI, and BstXI recognition sites in the CM region. In order to analyze enhancer functions of these partial fragments, the hsp-GFP plasmid shown in FIG. 4 was used to assay GFP expression. hsp-GFP was prepared by inserting the zebrafish heat shock protein (hsp) 70 promoter in a region upstream of the enhanced GFP (represented by “EGFP” in FIG. 4).

Partial fragments of the CM region were prepared by digesting with restriction enzymes XhoI, PstI, and BstXI. Partial fragments of the CM region were inserted into a immediate upstream of hsp in hsp-GFP to construct 8 types of plasmids, i.e., CMEX-hsp-GFP, CMX-hsp-GFP, CMXP-hsp-GFP, CMP-hsp-GFP, CMB1-hsp-GFP, CMB3-hsp-GFP, CMB4-hsp-GFP, and CMB2-hsp-GFP (FIG. 3).

Subsequently, these 8 types of plasmids were microinjected into one-cell stage zebrafish embryos, and expression of EGFP was assayed.

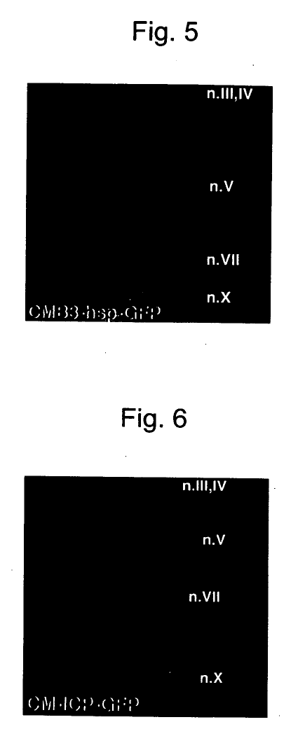

The assay revealed that fluorescence was emitted and EGFP was expressed when CMX-hsp-GFP, CMP-hsp-GFP, and CMB3-hsp-GFP had been injected. FIG. 5 is a photograph showing 60 hpf embryo to which CMB3-hsp-GFP had been microinjected. For the purpose of comparison, FIG. 6 is a photograph showing 60 hpf embryo to which CM-ICP-GFP prepared by inserting the entire CM region into immediate upstream of the ICP of ICP-GFP had been microinjected. In FIG. 5 and FIG. 6, “n.III” represents the oculomotor nerve, “n.IV” represents the trochlear nerve, “n.V” represents the trigeminal nerve, “n.VII” represents the facial nerve, and “n.X” represents the vagus nerve. As is apparent from FIG. 5 and FIG. 6, GFP expression was observed in the cell bodies and the axons of the cranial nerve, i.e., the oculomotor nerve, the trochlear nerve, the trigeminal nerve, the facial nerve, and the vagus nerve.

Based on the above results, a region of approximately 800 kbp in the CM region was designated as the minimal region exhibiting enhancer activity. An enhancer that is capable of improving the expression efficiency of a given gene in motor neurons was identified by determining the nucleotide sequence of this region of approximately 800 kbp (SEQ ID NO: 1). This region containing the enhancer was located approximately 20 kbp downstream of the transcription initiation site of the Islet-1 gene.

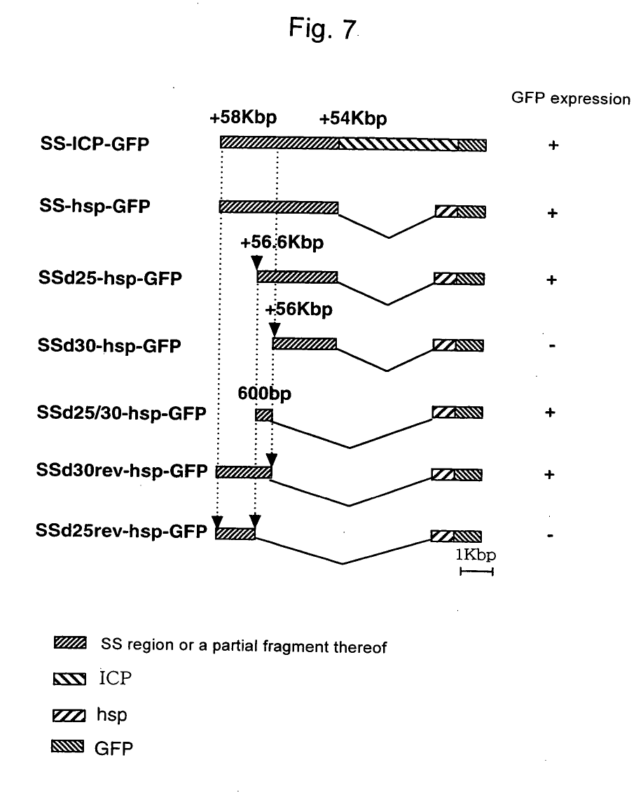

Since there is no adequate restriction enzyme recognition site in the SS region, a Kilo sequence deletion kit (Takara Shuzo Co., Ltd) was used to construct 5 types of plasmids having partial fragments lacking given regions, i.e., SSd25-hsp-GFP, SSd30-hsp-GFP, SSd25/30-hsp-GFP, SSd30rev-hsp-GFP, and SSd25rev-hsp-GFP, as shown in FIG. 7.

Subsequently, these 5 types of plasmids, the SS-hsp-GFP plasmid containing the entire SS region, and SS-ICP-GFP having the entire SS region inserted into immediate upstream of the ICP of ICP-GFP were microinjected into one-cell stage zebrafish embryos, and EGFP-induced fluorescence expression was assayed.

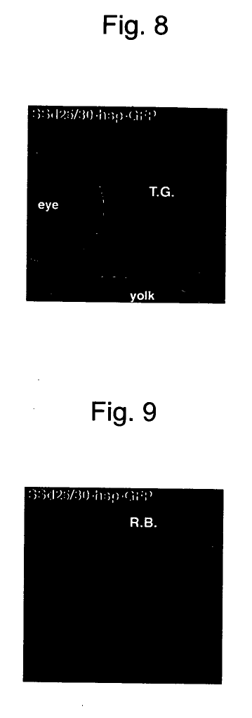

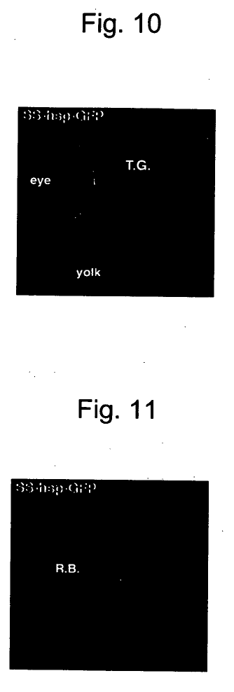

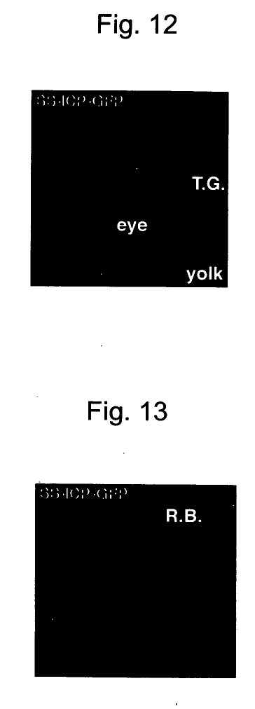

The assay revealed that fluorescence was emitted and EGFP was expressed when SSd25-hsp-GFP, SSd25/30-hsp-GFP, and SSd30rev-hsp-GFP had been injected,. FIG. 8 and FIG. 9 are lateral views showing 32 hpf embryo to which SSd25/30-hsp-GFP had been microinjected. For the purpose of comparison, FIG. 10 and FIG. 11 are lateral views showing 32 hpf embryo to which SS-hsp-GFP had been microinjected. For further comparison, FIG. 12 and FIG. 13 are lateral views showing 32 hpf embryo to which SS-ICP-GFP had been microinjected. In FIGS. 8 to 13, “T.G.” represents the trigeminal ganglion cells, “R.B.” represents the Rohon-Beard cell, “eye” represents an eye, and “yolk” represents an egg yolk.

From FIGS. 8, 10, and 12, GFP expression was observed in the trigeminal ganglion cells. In contrast, GFP expression was observed in the Rohon-Beard cell, which was a primary sensory neuron located in the spinal cord, from FIGS. 9, 11, and 13.

Based on the above results, a region of approximately 600 kbp in the SS region was designated as the minimal region exhibiting enhancer activity. An enhancer that was capable of improving the expression efficiency of a given gene in sensory neurons was identified by determining the nucleotide sequence of this region of approximately 600 kbp (SEQ ID NO: 5). This region containing the enhancer was located approximately 55 kbp downstream of the transcription initiation site of the Islet-1 gene.

### EXAMPLE 2

### Identification of Enhancer Derived from another Organism

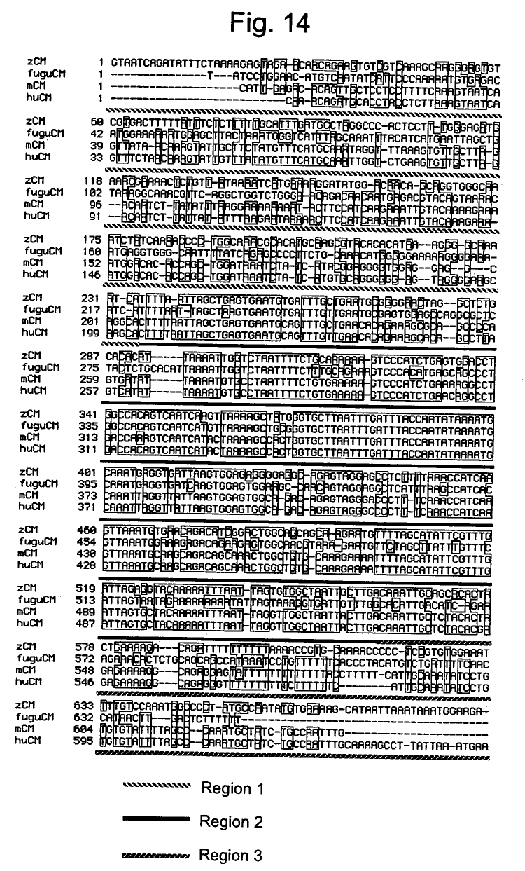

Based on the nucleotide sequence of an enhancer in motoneurons(hereafter referred to as “zCM,” SEQ ID NO: 1), a homology search was conducted using the NCBI database. BLAST was employed for a homology search. As a result, a sequence exhibiting a high level of homology (81%) to the nucleotide sequence as shown in SEQ ID NO: 1 over a region of approximately 230 bp was obtained from the human genome database (hereafter referred to as “huCM,” SEQ ID NO: 2). Also, sequences exhibiting high levels of homology to the nucleotide sequence as shown in SEQ ID NO: 1 were obtained from the mouse genome database and the fugu genome database (hereafter, referred to as “mCM” and “fuguCM;” SEQ ID NO: 3 and SEQ ID NO: 4, respectively). The results of a homology search among zCM, huCM, mCM, and fuguCM are shown in FIG. 14. As a result of examining the results of a homology search shown in FIG. 14, the sequences was divided into 3 regions based on degree of homology as follows. That is, region 2 exhibiting the highest homology and being highly conserved among different species; region 3 exhibiting the second-highest homology after region 2; and region 1 exhibiting the third-highest homology after region 3. Between zCM and huCM, homology in region 2 was 81%, that in region 3 was 63%, and that in region 1 was 53%.

Region 2 in zCM is equivalent to a nucleotide sequence consisting of nucleotides 235 to 560 of SEQ ID NO: 1. Region 2 in huCM is equivalent to a nucleotide sequence consisting of nucleotides 204 to 528 of SEQ ID NO: 2. Region 2 in mCM is equivalent to a nucleotide sequence consisting of nucleotides 206 to 530 of SEQ ID NO: 3. Region 2 in fuguCM is equivalent to a nucleotide sequence consisting of nucleotides 211 to 555 of SEQ ID NO: 4. Since region 2 is highly conserved among different species, it is suggested to be the most important region for enhancer functions.

Subsequently, primers were designed for huCM and mCM so as to contain sequences in the vicinity thereof, and each genome DNA was amplified by PCR and then isolated. Specifically, the huCM forward primer and the huCM reverse primer were designed in order to amplify huCM from the human genome. The sequences are as shown below.

| | |
|---|---|
| HuCM forward: | |
| 5′-GGGAATTCAAACAGATGCACCTACCTC-3′ | (SEQ ID NO: 9) |
| HuCM reverse: | |
| 5′-GGGAATTCGGACATATGGCTAGAGTGTG-3′ | (SEQ ID NO: 10) |

EcoRI restriction enzyme recognition sequences were added to the above primers at both ends for later construction of plasmid constructs.

Similarly, the mCM forward primer and the mCM reverse primer were designed in order to amplify mCM from the mouse genome. The sequences are as shown below.

| MCM forward: | |
|---|---|
| 5′-GGGAATTCATTGAGACACAGTTGCTCCTCC- | (SEQ ID NO: 11) |
| 3′ | |
| MCM reverse: | |
| 5′-GGGAATTCAAATTGGCAGATAGCATTTGGG- | (SEQ ID NO: 12) |
| 3′ | |

These primers were used to amplify genome fragments (huCM and mCM) from human and mouse genomes by a standard PCR technique. The amplified products were subcloned into the pGEMT easy vector (Promega) via TA cloning and then isolated. A large quantity of plasmids containing the isolated genome fragments (huCM and mCM) were prepared by the alkaline-SDS method and digested with the EcoRI restriction enzyme. The genome fragments of interest (huCM and mCM) were then collected via agarose gel electrophoresis. The collected genome fragments (huCM and mCM) were subcloned into upstream of the ICP of ICP-GFP that had been digested with the EcoRI restriction enzyme. Thus, huCM-ICP-GFP and mCM-ICP-GFP plasmids were constructed.

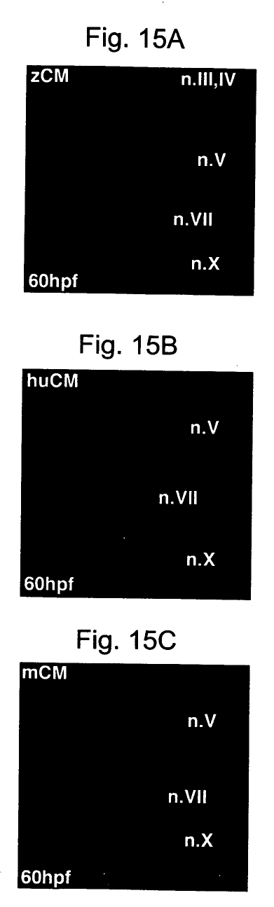

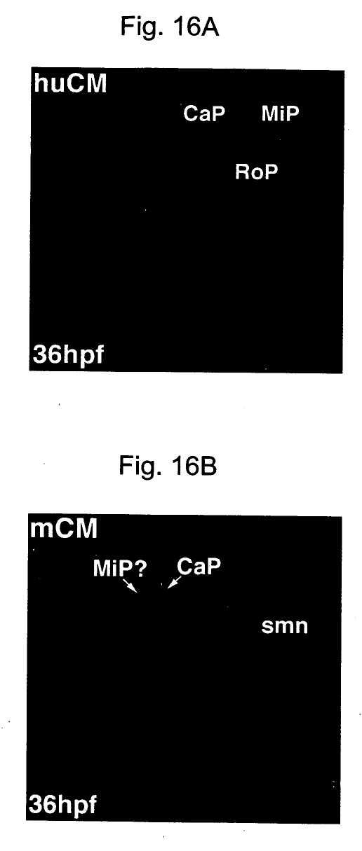

Subsequently, the obtained huCM-ICP-GFP and mCM-ICP-GFP plasmids were microinjected into a one-cell stage zebrafish embryo and GFP expression was assayed. The results are shown in FIG. 15 and FIG. 16. In FIG. 15, A is a dorsal view of the head of 60 hpf embryo microinjected with zCM-ICP-GFP; B is a dorsal view of the head of 60 hpf embryo microinjected with huCM-ICP-GFP; and C is dorsal view of the head of 60 hpf zebrafish embryo microinjected with mCM-ICP-GFP. In FIG. 15, “n.III” represents the oculomotor nerve, “n.IV” represents the trochlear nerve, “n.V” represents the trigeminal nerve, “n.VII” represents the facial nerve, and “n.X” represents the vagus nerve.

In FIG. 16, A is a photograph showing a lateral view of the spinal cord when huCM-ICP-GFP is used and B is a photograph showing a side view of the spinal cord when mCM-ICP-GFP is used. In FIG. 16, “CaP” represents a primary motoneuron that is referred to as “caudal primary,” “MiP” represents a primary motoneuron that is referred to as “middle primary,” “RoP” represents a primary motoneuron that is referred to as “rostral primary,” and “smn” represents a secondary motoneuron. RoP, MiP, and CaP are primary motor neurons that are present in quantities of 3 each in each hemi segment of the zebrafish embryo and are known to extend axons in the septal, dorsal, and ventral areas of the trunk, respectively. The roles thereof have not yet been elucidated; however, they are considered to play key roles in guiding extension of axons in the secondary motor neurons that finally control muscles of the mesodermal somite.

As is apparent from FIG. 15, when huCM and mCM were employed, GFP expression was observed in the cell bodies and the axons of the cranial nerve, i.e., the oculomotor nerve, the trochlear nerve, the trigeminal nerve, the facial nerve, and the vagus nerve, as is the case where zCM had been employed. Thus, huCM and mCM were also verified to exhibit enhancer activities for improving gene expression efficiency in motor neurons.

Unlike the case where zCM-ICP-GFP was used, GFP expression was observed in the spinal motor neurons only when mCM-ICP-GFP and huCM-ICP-GFP were employed, according to FIG. 16. This clearly shows that enhancer functions for improving gene expression efficiency were exhibited particularly in the spinal motor neurons among various types of motor neurons when huCM and mCM were employed.

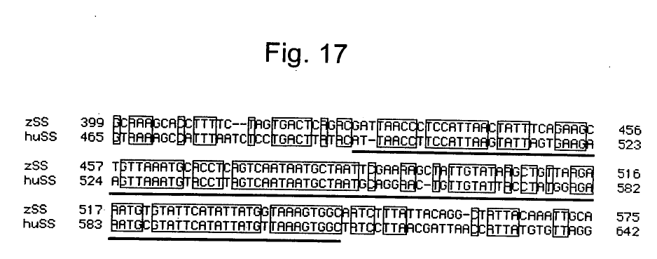

Based on the nucleotide sequence of an enhancer in sensory neurons (hereafter referred to as “zSS;” SEQ ID NO: 5), a homology search was conducted using the NCBI database. BLAST was employed for a homology search. As a result, a sequence exhibiting a high level of homology (80%) to the nucleotide sequence as shown in SEQ ID NO: 5 over a region of approximately 120 bp was obtained from the human genome database (hereafter referred to as “huSS;” SEQ ID NO: 6). The results of a homology search between zSS and huSS are shown in FIG. 17.

As a result of examining the results of a homology search shown in FIG. 17, the following was found. That is, 1) huSS does not have any apparent repeated sequence even though it was compared with the entire sequence of the roughly analyzed human genome; 2) huSS and zSS are highly homologous over a relatively long sequence (e.g., the region between nucleotides 378 and 553 of SEQ ID NO: 5 and the region between nucleotides 178 and 353 of SEQ ID NO: 6); and 3) huSS is located relatively close to the human Islet-1 gene. Accordingly, the fragment can be considered to have enhancer activities in sensory neurons.

### EXAMPLE 3

### Preparation of Transgenic Animal 1

1) Transgenic Zebrafish

At the outset, the zCM-ICP-GFP, huCM-ICP-GFP, mCM-ICP-GFP, zSS-ICP-GFP, and huSS-ICP-GFP plasmids prepared in Example 2 were treated with NotI to cleave at sites upstream of the enhancer regions to prepare linear plasmids. Subsequently, linear plasmids were microinjected into single cell zebrafish embryos. In this case, linear plasmids were prepared so as to adjust the DNA concentration level to 50 ng/microlitter.

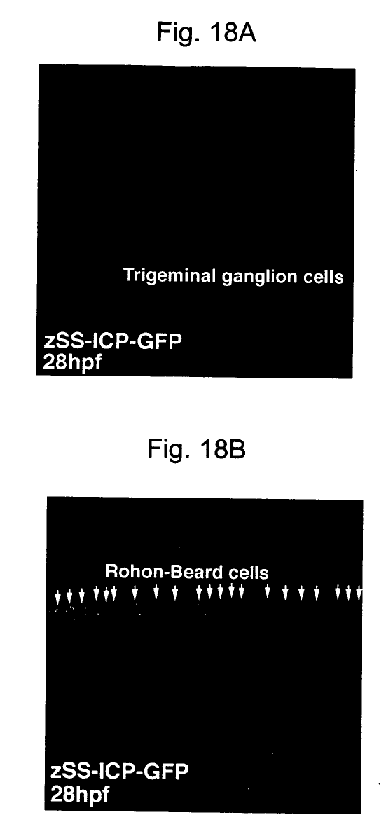

Thereafter, individuals exhibiting potent GFP expression in 24 hpf embryos were selected and allowed to grow to adult fish (F0 generation). The resulting adult fish were subjected to mating, and the F1 generation thereof was screened under a fluorescence microscope. Among the obtained transgenic zebrafish, the zSS-ICP-GFP transgenic zebrafish was selected, and a confocal image (LSM 510, Zeiss) thereof is shown in FIG. 18. FIG. 18 A shows the trigeminal neuron and FIG. 18 B shows the Rohon-Beard cell in the spinal cord. As is apparent from FIG. 18, GFP expression was observed in the sensory neurons, i.e., the trigeminal neuron and the Rohon-Beard cell in the spinal cord, of the zSS-ICP-GFP transgenic zebrafish.

2) Transgenic Mouse

The GFP reporter gene was exchanged with human placental alkaline phosphatase (PLAP) when a transgenic mouse is prepared. PLAP is a GPI-linked protein that is very efficiently transferred onto a membrane upon expression thereof in neurons and is able to effectively stain the axons. While endogenous alkaline phosphatase of a mouse is sensitive to heat, PLAP is heat-resistant. Thus, PLAP activity can be selectively detected via heat treatment. Thus, use of a PLAP reporter gene for preparation of a transgenic mouse was determined.

At the outset, a basic vector (ICP-PLAP) having the PLAP gene located in the downstream of the ICP was prepared. Specifically, the PLAP gene was amplified and isolated from the Z/AP vector (provided by Dr. Corrinne G. Lobe of Sunnybrook & Women's College Health Sciences Center) via a standard PCR technique. Primers for this PCR operation (PLAP 5 prime, PLAP 3 prime) were designed so that the restriction enzyme KpnI recognition sequence would be on the 5′ side and the BglII recognition sequence would be on the 3′ side. The sequences are as shown below.

| | |
|---|---|
| PLAP 5 prime: | |
| GGTACCCTGCCTCGCCACTGTCCTGC | (SEQ ID NO: 13) |
| PLAP 3 prime: | |
| AGATCTCAGGGAGCAGTGGCCGTCTCC | (SEQ ID NO: 14) |

Subsequently, the PCR-amplified DNA fragment was subcloned into the pGEMT easy vector, this vector was digested with KpnI and BglII restriction enzymes to cleave the PLAP gene from the vector, and the PLAP genes were collected by agarose gel electrophoresis. The collected PLAP genes were subcloned into the KpnI and BamHI restriction enzyme recognition sites of the pBluescript SKII vector that has been prepared by inserting the intron/poly A additional signals derived from the SV 40 virus into the BamHI and XbaI restriction enzyme recognition sites. A large quantity of plasmids comprising the PLAP genes and the intron/poly A additional signals derived from the SV 40 virus were prepared by the alkaline-SDS method. The PLAP gene and the intron/poly A additional signal derived from the SV 40 virus were extracted from the vector using KpnI and XbaI restriction enzymes and collected by agarose gel electrophoresis. The collected PLAP genes and the intron/poly A additional signal derived from the SV 40 virus were inserted into downstream of the ICP of the ICP plasmid that has been digested with the KpnI and XbaI restriction enzymes in the same manner. The intron/poly A additional signals derived from the SV 40 virus were derived from pcDNA1 (Invitrogen). Subsequently, zCM and zSS were inserted immediate upstream of ICP (promoter) of ICP-PLAP as with the case of construction of the ICP-GFP plasmid.

The plasmid prepared by inserting zCM into ICP-PLAP was designated as zCM-ICP-PLAP, and the plasmid prepared by inserting zSS into ICP-PLAP was designated as zSS-ICP-PLAP.

Subsequently, the prepared zCM-ICP-PLAP and zSS-ICP-PLAP were subjected to mass production by the alkaline-SDS method. Thereafter, the mass-produced zCM-ICP-PLAP and zSS-ICP-PLAP were digested with the NotI and XbaI restriction enzymes to extract zCM-ICP-PLAP and zSS-ICP-PLAP from the vector. The extracted zCM-ICP-PLAP and zSS-ICP-PLAP were adjusted to a final concentration of 30 ng/microlitter. The collected zCM-ICP-PLAP and zSS-ICP-PLAP were microinjected into fertilized eggs of a B6 mouse, and the injected fertilized eggs were transplanted into the oviductus of a pseudopregnant mouse. Genomic DNA was extracted from the tail of weaned offspring, and transgene-positive individuals were selected by PCR.

Among the selected transgene-positive individuals (the offspring of the F0 generation (P0)), stillborn individuals were subjected to alkaline phosphatase staining. More specifically, such individuals were soaked in a phosphate buffer and then beheaded, followed by extraction of the brains with the use of a microforceps. Visceral organs were removed from the trunks, and the trunks were decorticated to expose musculoskeletons. The extracted brains and the trunks were immobilized with the use of 4% paraformaldehyde at room temperature for 3 hours and then rinsed with a phosphate buffer. After thorough rinsing, heat treatment was carried out (at 72° C. for 45 minutes) in order to deactivate endogenous alkaline phosphatase. Thereafter, the products were washed two times with a coloring buffer (100 mM Tris, pH=9.5, 100 mM NaCl, 50 mM MgCl₂) and then colored with the use of BM purple (Roche).

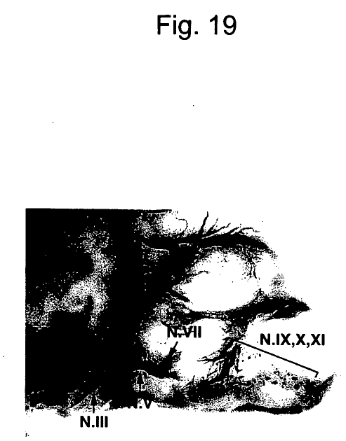

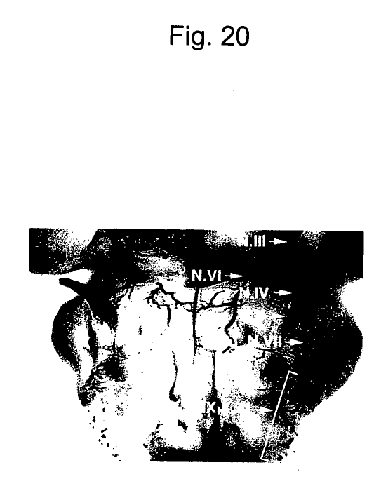

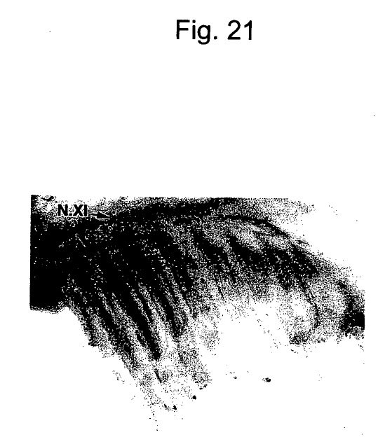

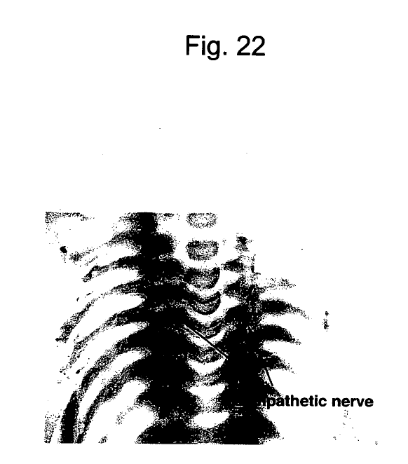

FIG. 19 to FIG. 22 show the results of imaging each regions of the transgene-positive individuals (stillborns) prepared with the use of zCM-ICP-PLAP. FIG. 19 is a photograph showing a side view of the head, FIG. 20 is a photograph showing the anterior view of the brain, FIG. 21 is a photograph showing a lateral view of ribs, and FIG. 22 is a photograph showing the ribs from the inside. In FIG. 19 to FIG. 22, “N.III” represents the oculomotor nerve, “N.IV” represents the trochlear nerve, “N.V” represents the trigeminal nerve, “N.VI” represents the oculomotor nerve, “N.VII” represents the facial nerve, “N.IX” represents the hypoglossal nerve, “N.X” represents the vagus nerve, and “N.XI” represents the accessory nerve, “sympathetic nerve” represents the sympathetic nerve. From the photographs shown in FIG. 19 to FIG. 22, the axons of the cranial nerves and those in the intercostal nerves and the sympathetic trunks were found to have been stained in the transgene-positive individuals prepared with the use of zCM-ICP-PLAP.

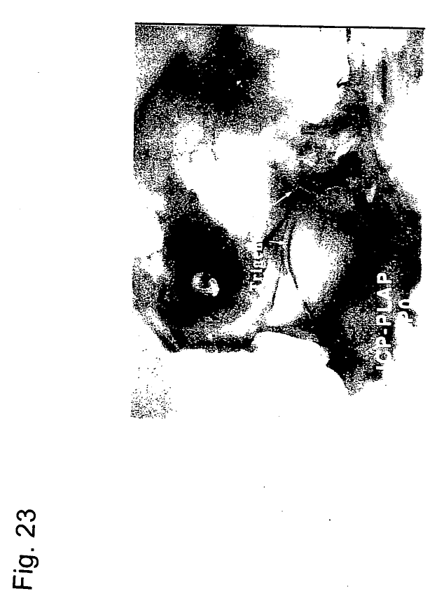

FIG. 23 shows the results of imaging each regions of the transgene-positive individuals (stillborns) prepared with the use of zSS-ICP-PLAP. FIG. 23 is a photograph showing a lateral view of the brain. In FIG. 23, a position represented by “trigeminal sensory” indicated by an arrow is the axon of the sensory branch of the trigeminal nerve. From the photograph shown in FIG. 23, the axon of the sensory branch of the trigeminal nerve was found to have been stained in the transgene-positive individuals prepared with the use of zSS-ICP-PLAP.

### EXAMPLE 4

### Preparation of Transgenic Animal 2

In this example, localization of enhancer activity in the CM region and that in the SS region were further studied. At the outset, SS-hsp-GFP prepared in Example 2 (a vector having the entire SS region inserted directly above hsp of hsp-GFP) was used to prepare a transgenic zebrafish in the same manner as in Example 3 in order to study localization of enhancer activity in the SS region. Also, transgenic mice were prepared in the same manner as in Example 3 with the use of zSS-ICP-PLAP and huSS-ICP-PLAP.

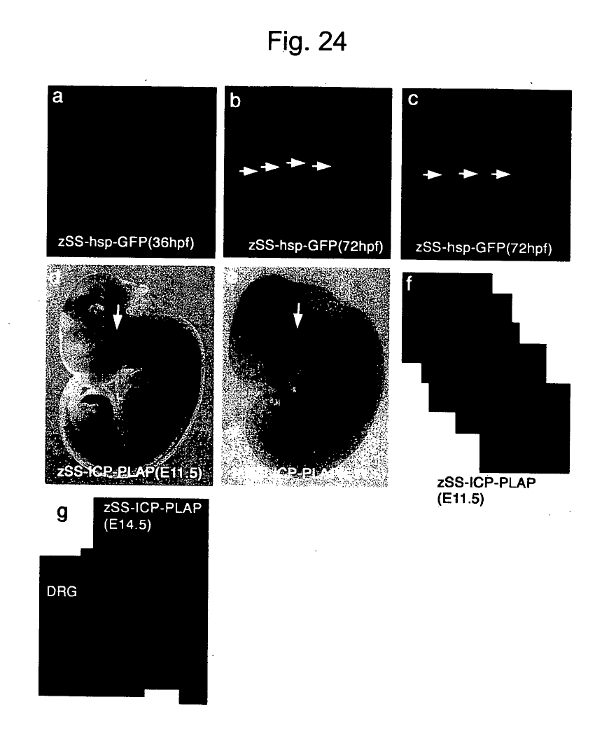

The results of imaging each regions in the transgenic zebrafish prepared with the use of SS-hsp-GFP are shown in FIG. 24 a, b, and c. FIG. 24 a shows the trigeminal ganglion cells, FIG. 24 b and c show the abductor muscle of pectoral fin and the ventral trunk muscles, respectively. In FIG. 24 b and c, an arrow represents the direction of the axon.

The results of imaging each regions in the transgenic mouse prepared with the use of zSS-ICP-PLAP are shown in FIG. 24 d, f, and g. FIG. 24 d is a photograph showing a lateral view of the entire transgenic mouse (E11.5), FIG. 24 f is a photograph showing the cervical spinal cord of the transgenic mouse (E11.5), and FIG. 24 g is a photograph showing the cervical spinal cord of the transgenic mouse (E14.5). FIG. 24 e is a photograph showing the lateral view of the transgenic mouse (E11.5) prepared with the use of huSS-ICP-PLAP. In FIG. 24 d and e, an arrow indicates the trigeminal ganglion cells. In FIG. 24, “DRG” represents the dorsal root ganglion cells.

As shown in FIG. 24 a and b, reporter gene (GFP) expression was observed in the trigeminal ganglion cells and in the motor neurons that extended axons ventrally in the transgenic zebrafish prepared with the use of zSS-hsp-GFP. As shown in FIG. 24 d, f, and g, reporter gene (PLAP) expression was observed in the trigeminal ganglion cells (FIG. 24 d), in the motor neurons (FIG. 24 f) that extended axons into the ventral half of the limb bud, and in the dorsal root ganglion cells (FIG. 24 g).

In contrast, reporter gene (PLAP) expression was not observed in the trigeminal neurons of the transgenic mouse prepared with the use of huSS-ICP-PLAP as shown in FIG. 24 e. However, reporter gene (PLAP) expression was observed in the motor neurons that extended axons ventrally.

The above results demonstrated that both the enhancer activity in the zSS and huSS were found to drive gene expression in the motor neurons that extended axons ventrally. In contrast, the sensory neuron-specific enhancer activity was only found in zSS, and not in huSS.

Subsequently, CM-ICP-GFP prepared in Example 2 was used to generate a transgenic zebrafish in the same manner as in Example 3 in order to study localization of enhancer activity in the CM region. Also, a transgenic mouse was prepared in the same manner as in Example 3 with the use of huCM-ICP-PLAP.

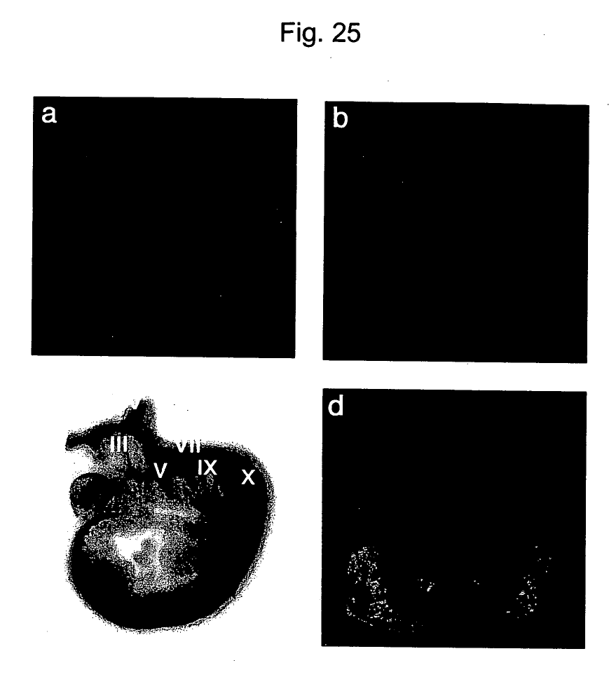

FIG. 25 a shows the results of imaging the spinal cord of the 72 hpf transgenic embryo generated with the use of a larger zebrafish genome fragment containing zCM. In FIG. 25 b, c, and d, the results of imaging each location of the transgenic mouse prepared with the use of huCM-ICP-PLAP are shown. FIG. 25 b is a photograph showing the thoracic spinal cord in a frozen section of the transgenic mouse (E12.5) that was doubly stained with the anti-Islet-1 antibody (green) and with the anti-PLAP antibody (red). FIG. 25 c is a photograph showing a lateral view of the entire transgenic mouse (E10.5). FIG. 25 d is a photograph showing the thoracic spinal cord in a frozen section of the transgenic mouse (E11.5) that was doubly stained with the anti-Islet-1 antibody (green) and with the anti-PLAP antibody (red).

As shown in FIG. 25 a, reporter gene (GFP) expression was observed in motor neurons that extended axons dorsally among the spinal secondary motor neurons in the transgenic zebrafish. As shown in FIG. 25 b, reporter gene (PLAP) expression was observed in motor neurons that extended axons dorsally in the transgenic mouse prepared with the use of huCM-ICP-PLAP. As shown in FIG. 25 c and 25 d, reporter gene (PLAP) expression was transiently observed in all the motor cranial nerve nuclei, in the spinal motoneurons, and in the Islet-1 positive motor neurons of the transgenic mouse generated with the use of huCM-ICP-PLAP.

Thus, the huCM region contains the enhancer activity transiently for all the motor neurons and the enhancer activity specifically for motor neurons that extended axons dorsally.

As is apparent from this example, the SS enhancers and the CM enhancers exhibited enhancer activities specific to different types of motor neurons.

All publications, patents, and patent applications cited herein are incorporated herein by reference in their entirety.

### INDUSTRIAL APPLICABILITY

As is apparent from the foregoing description, the present invention can provide a novel enhancer that can improve the expression efficiency of a gene of interest in motor neurons and/or in sensory neurons. Also, the present invention can provide a vector comprising such enhancer, a transgenic cell line, a transgenic animal, a method for regulating gene expression, a method for evaluating differentiation of pluripotent stem cells, and a method for regenerating motor neurons and/or sensory neurons.

1. An enhancer consisting of the following DNA (a), (b), or (c):
(a) DNA consisting of the nucleotide sequence as shown in any one of SEQ ID NOs: 1 to 4;
(b) DNA consisting of a nucleotide sequence derived from the nucleotide sequence as shown in any one of SEQ ID NOs: 1 to 4 by deletion, substitution, or addition of one or more nucleotides and capable of enhancing gene expression efficiency in motor neurons; or
(c) DNA consisting of a nucleotide sequence capable of hybridizing under stringent conditions to a nucleotide sequence complementary to the nucleotide sequence as shown in any one of SEQ ID NOs: 1 to 4 and capable of enhancing gene expression efficiency in motor neurons.

2. An enhancer consisting of the following DNA (a), (b), or (c):
(a) DNA consisting of any one of a nucleotide sequence consisting of nucleotides 235 to 560 of SEQ ID NO: 1, a nucleotide sequence consisting of nucleotides 204 to 528 of SEQ ID NO: 2, a nucleotide sequence consisting of nucleotides 206 to 530 of SEQ ID NO: 3, or a nucleotide sequence consisting of nucleotides 211 to 555 of SEQ ID NO: 4;
(b) DNA consisting of a nucleotide sequence derived from any one of a nucleotide sequence consisting of nucleotides 235 to 560 of SEQ ID NO: 1, a nucleotide sequence consisting of nucleotides 204 to 528 of SEQ ID NO: 2, a nucleotide sequence consisting of nucleotides 206 to 530 of SEQ ID NO: 3, or a nucleotide sequence consisting of nucleotides 211 to 555 of SEQ ID NO: 4 by deletion, substitution, or addition of one or more nucleotides and capable of enhancing gene expression efficiency in motor neurons; or
(c) DNA consisting of a nucleotide sequence capable of hybridizing under stringent conditions to a nucleotide sequence complementary to any one of a nucleotide sequence consisting of nucleotides 235 to 560 of SEQ ID NO: 1, a nucleotide sequence consisting of nucleotides 204 to 528 of SEQ ID NO: 2, a nucleotide sequence consisting of nucleotides 206 to 530 of SEQ ID NO: 3, or a nucleotide sequence consisting of nucleotides 211 to 555 of SEQ ID NO: 4 and capable of enhancing gene expression efficiency in motor neurons.

3. The enhancer according to claim 1, wherein the motor neurons dorsally extend axons.

4. An enhancer consisting of the following DNA (a), (b), or (c):
(a) DNA consisting of the nucleotide sequence as shown in SEQ ID NO: 5;
(b) DNA consisting of a nucleotide sequence derived from the nucleotide sequence as shown in SEQ ID NO: 5 by deletion, substitution, or addition of one or more nucleotides and capable of enhancing gene expression efficiency in sensory neurons; or
(c) DNA consisting of a nucleotide sequence capable of hybridizing under stringent conditions to a nucleotide sequence complementary to the nucleotide sequence as shown in SEQ ID NO: 5 and capable of enhancing gene expression efficiency in sensory neurons.

5. An enhancer consisting of the following DNA (a), (b), or (c):
(a) DNA consisting of the nucleotide sequence as shown in SEQ ID NO: 5 or 6;
(b) DNA consisting of a nucleotide sequence derived from the nucleotide sequence as shown in SEQ ID NO: 5 or 6 by deletion, substitution, or addition of one or more nucleotides and capable of enhancing gene expression efficiency in motor neurons that ventrally extend axons; or
(c) DNA consisting of a nucleotide sequence capable of hybridizing under stringent conditions to a nucleotide sequence complementary to the nucleotide sequence as shown in SEQ ID NO: 5 and capable of enhancing gene expression efficiency in motor neurons that ventrally extend axons.

6. A vector comprising the enhancer according to claim 1.

7. The vector according to claim 6 further comprising a promoter and a gene comprising a coding region.

8. A transgenic cell line comprising the vector according to claim 6.

9. A transgenic animal comprising the vector according to claim 6.

10. A method for regulating gene expression, wherein expression efficiency of a given gene is improved under the control of the enhancer according to claim 1.

11. A method for regulating gene expression comprising a step of introducing a nucleic acid construct comprising the enhancer according to claim 1 and the given gene into cells, wherein expression efficiency of the given gene is improved in motor neurons and/or in sensory neurons.

12. A method for evaluating differentiation of pluripotent stem cells comprising a step of introducing a nucleic acid construct comprising the enhancer according to claim 1, a promoter, and a reporter gene into pluripotent stem cells and a step of inducing the pluripotent stem cells to differentiate, wherein reporter gene expression is assayed to evaluate whether or not the pluripotent stem cells are differentiated into motor neurons or sensory neurons.

13. A method for regenerating motor neurons/sensory neurons comprising steps of:
introducing a nucleic acid construct comprising the enhancer according to claim 1, a promoter, and a reporter gene into pluripotent stem cells;
inducing the pluripotent stem cells to differentiate;
assaying reporter gene expression to evaluate whether or not the pluripotent stem cells are differentiated into motor neurons or sensory neurons to thereby select motor neurons or sensory neurons; and
transplanting the selected motor neurons or sensory neurons.


SEQUENCE LISTING (14 sequences)

SEQ ID NO: 1 - DNA, 820 bp, Dana rerio
gtaatcagatatttctaaaagagtagaacaacagaagtgtcgtcaaagcaagggagtgtcgtgactttttatttctctttttgcatttgatgcctaggcccactcctttgggagatgaaacgaaaactctgttataaaatcatgaaaaggatatggacaacagcaggtgggcaaatctatcaaaacccctggcaaacgcacatgcaagcgtacacacataaaggggcaaaatcattttaattagctgagtgaatgtgatttgctgaatgcggggaactaggctctgcacacattaaaattggtctaattttctgcaaaaaagtcccatctgagtggacctggccacagtcaatcaagttaaaagctatgggtgcttaatttgatttaccaatataaaatgcaaatgaggtgattaagtggagaggggaggcagagtaggagcctcttttaaaccatcaagttaaatgtgaacagacatcggactggcagcagcaagaatgttttagcatattcgtttgattagaggtacaaaaatttaattagtgtggctaattgcttgacaaattgcagcacactactgaaaagacagattttttttttaaaaccgtgcaaaaccccctccgtgtggaaattttgtccaaatggcccctatgccaatatgtgaaaagcataattaaataaatggaagatggcacaacagtaccttacaatagcaaatgagataattgcctgtaattaggtgggacacaagtctatgtccatatgtcgtgtttctcttcagctactctccgttcctctcgtaggacaaatctaataagcc

SEQ ID NO: 2 - DNA, 725 bp, Homo sapiens
caaacagatgcacctacctcttaaagtaatcagtttctaacaaagtattgtttatatgtttcatgcaaattggtctgaagtgttgcttagacaatcttattatatttaagaataaaaacttccatcaagaaattgtacaaagaaaatggacacaccagctggataaatctatcatgtgcaggggggagtaggggaagcaagcacttttaattagctgagtgaatgcagtttgttgaacacagaaagcacagccttagtcatattaaaatgtgcctaattttctgtgaaaaagtcccatctgaacaggcctgaccacagtcaatcatactaaaagccactggtgcttaatttgatttaccaatataaaatgcaaattaggttattaagtggagtggcagacagagtagggcccctttcaaaccatcaagttaaatgcaagcagacagcaaactggctgtgcaaagaaaattttagcatattcgtttgattagtgctacaaaaatttaattaggttggctaattacttgacaaattgctctacacgagagaaaaggcagagggttttttttttcttttttcattgcaaatatcctgtgtgtattttagcccaaatgctatctgccaatttgcaaaagccttattaaatgaatggaagatggtccccaataatagcaaatcatataatgcatataattagacaaggcccacactctagccatatgtcc

SEQ ID NO: 3 - DNA, 638 bp, Mus musculus
cattgagacacagttgctcctccttttcaaagtaatcagttataacaaagtattgcttctatgtttcatgcaaataggtttaaagtgttgcttagacaatcttatatttaaggaaaaaaaatacttccatcaagaaattgtacaaaagaaaatggacacaccagctggataaatctatcatacggaggggtggaggaggcaggcacttttaattagctgagtgaatgcagtttgctgaacacagaaagcgcagccccagtgatattaaaatgtgcctaattttctgtgaaaaagtcccatctgaaaaggcctgaccaaagtcaatcatactaaaagccactggtgcttaatttgatttaccaatataaaatgcaaattaggttattaagtggagtggcagacagagtagggaccctttcaaaccatcaagttaaatgcaagcagacagcaaactggctgtgcaaagaaaattttagcatattcgtttgattagtgctacaaaaatttaattaggttggctaattacttgacaaattgctctacactagagaaaaggcagaggagtatttttttttttttacctttttcattgcaaatatcctgtgtgtattttagcccaaatgctatctgccaatttg

SEQ ID NO: 4 - DNA, 650 bp, Fugu rubripes
tatcctggaacatgtcaatatcattcccaaaaatgtgagacatggaaaaaatggagcttactaaatgggtcatttagcaaatttacatcatgaattagctgtaaaggcaaacgttcaggctggtctgggaacagacaacaatgagacgtacagtaaaacatgaggtgggcaattttatcagagccccttctgcaaacatgggggaaaaaggggaaaatcattttaattagctaagtgaatgtgatttgttgaatgcgagtggagccaggcgctctactctgcacattaaaattggtctaattttctttgcagaaagtcccacatgagcagccctggccacagtcaatcatgttaaaagctgcgggtgcttaatttgatttaccaatataaaatgcaaatgaggtgatcaagtggagtggaagccaacagtaggaggctcatttaagccatcacgttaaatggaaagagacagaagagtggcaacgtaaagaatgttctagcttatttgtttcattagtaatagaaaaaaaaatattagtaaaggtgattgtttggcacattgacatcagaaagaaacactctgcagcagccataaatcctgttttttcaccctacatgtctgattttcaaccataacttgactctttttt

SEQ ID NO: 5 - DNA, 636 bp, Dana rerio
gtgcagctttagacatttaaaattgtcttcacctatcaattaggtaattttttcggctcttaaatgtctcattttataggttttgcaggaatatgtacacttttcaagaaaaacataattaaaatgtgttaatttccatttaacaagcagtgtttagattatataatgcatcaataaactaactgtcatcactttctataaataaactattatcctcctaagccacatttactgggcaatgatcgattcatcatttcctatacagtataggctcagcagtccttcacatgtgtttgcgtattcaggaaatatatatcgaaggaaaggaacagagatacatttatctaatcgtcctctgaacaccccagcacactgtgtaatcaataaacttgttttaggcaaagcaccttttctagtgactcagacgattaaccctccattaactatttcagaagctgttaaatgcacctcagtcaataatgctaattcgaaaagctattgtataagctgttaagaaatgtgtattcatattatggtaaagtggcaatctttattacaggctattacaaattgcaaaaaaagtcaatatgtgaggggagatatttcacaccgtggtgaattatggtgctggaattc

SEQ ID NO: 6 - DNA, 456 bp, Homo sapiens
cgagggtgtcttcatcacttccatatcattgccttaaaaggcactgaccagaagaagcagatgacctcatttcaaaattattacagtacagagaactcagtttcaacattataattcattttatcatggaatatttcaaatttattatcagtttcctaacacataatggttaatcgttaaggatagccactttaacataatatgaatacgcatttctccataggtaatacaacagttcctgcattagcattattgactaaggtacatttaacttcttcactaatacttaatggaaggttaatgtataagtcaggagattaaatggcttttacttaaaacaagtatattgattaaaataacttagtgagatttttaaggttgatgatataaaaacagtcacacattttaatattttatttaatattaagagcaaattggagggtgcaacagatcaag

SEQ ID NO: 7 - DNA, 26 bp, Artificial Sequence
  primer
gaattcggatccaaggtcttcagtct

SEQ ID NO: 8 - DNA, 22 bp, Artificial Sequence
  primer
ggtacctgtattgatgggccac

SEQ ID NO: 9 - DNA, 27 bp, Artificial Sequence
  primer
gggaattcaaacagatgcacctacctc

SEQ ID NO: 10 - DNA, 28 bp, Artificial Sequence
  primer
gggaattcggacatatggctagagtgtg

SEQ ID NO: 11 - DNA, 30 bp, Artificial Sequence
  primer
gggaattcattgagacacagttgctcctcc

SEQ ID NO: 12 - DNA, 30 bp, Artificial Sequence
  primer
gggaattcaaattggcagatagcatttggg

SEQ ID NO: 13 - DNA, 26 bp, Artificial Sequence
  primer
ggtaccctgcctcgccactgtcctgc

SEQ ID NO: 14 - DNA, 27 bp, Artificial Sequence
  primer
agatctcagggagcagtggccgtctcc